(19) United States
(12) Patent Application Publication
(10) Pub. No.: US 2026/0215723 A1
(43) Pub. Date: Jul. 30, 2026

(54) DEVICE AND METHOD FOR DETERMINING A SEVERITY OF FOG

(71) Applicant: Fortis Medical Devices Teoranta, Galway (IE)

(72) Inventors: Gearóid Ólaighin, Galway (IE); Evismar Almeida de Andrade, Galway (IE)

(21) Appl. No.: 19/041,261

(22) Filed: Jan. 30, 2025

(51) Int. Cl. A61B 5/00; G16H 40/63; G16H 50/20

(52) U.S. Cl. CPC A61B 5/4082; A61B 5/742; A61B 5/7475; G16H 40/63; G16H 50/20

(57) ABSTRACT

A device for determining the severity of Freezing Of Gait (FoG) in a patient is provided. The device comprises a FoG registration interface comprising two thumb-actuated buttons for registering the FoG status of the patient as “FoG” (experiencing FoG) or “no FoG” (not experiencing FoG), where one button indicates “FoG” and the other button indicates “no FoG”. While a patient conducts a gait sequence, the device begins by setting the FoG status to “no FoG”; sets the FoG status to “FoG” when a user actuates the “FoG” button; sets the FoG status “no FoG” to when a user actuates the “no FoG” button, and repeats this for each change in the patient's FoG status; and determines the amount of time the FoG status of the patient was registered as FoG as a percentage of total time for the predefined gait sequence.

### TECHNICAL FIELD

The present disclosure relates generally to devices and methods for determining a severity of Freezing Of Gait (FoG) in a patient.

### BACKGROUND

Gait disturbances/artefacts are a common feature of neurological diseases such as e.g. Parkinson's disease (PD), Huntington's disease, Multiple sclerosis (MS) and stroke. Gait disturbances/artefacts can also be present due to mechanical wear and tear (for a variety of reasons) of the muscles, tendons, ligaments and joints, giving rise to joint stiffness, gait unsteadiness and gait variability. These gait disturbances/artefacts may have a significant impact on daily life, giving rise to reduced mobility and increased fall risk, and often lead to social isolation, falls and hospitalisation. Thus, there are both physical and mental health consequences of these gait disturbances, and their clinical management can be a major priority.

Gait disturbances may include episodes called Freezing Of Gait (FoG), characterized by a rapid onset and variable duration (typically <1 min) causing a breakdown in gait fluency. FoG can appear on the initiation of gait, during turning, during walking in narrow spaces, or randomly within the gait cycle, and is often medication refractory, leading to it being a major source of disability for patients affected by neurological disease. The biological mechanisms underlying FoG are yet to be fully elucidated. Attempting to walk in narrow passageways, adjusting one's stride length when reaching a destination, turning and stressful situations are common triggers for FoG.

Evidence from a range of studies suggests that cueing methodologies can improve gait performance. These cues may be temporal, spatial, audio or visual stimuli, which facilitate the initiation and continuation of repetitive sequential movements such as gait and can assist in handling, or even overcoming, gait irregularities, in particular freezing of gait episodes. Cues are normally intended to provide supportive information to the patient about step frequency or step amplitude leading to a change in the motor response of the subject. Some devices have been developed that provide cues as a means to stop gait irregularities. Other devices have been developed that provide cues as a means to prevent gait irregularities occurring in the first place. Such devices are activated (manually or automatically) when motion is detected, and provide continuous cueing, by for example repetitive sound from a metronome being delivered via an earpiece. More advanced systems aim to assess user cadence and adapt the delivered cue rhythm to the user's normal gait. An example of such a system is described in US201214124752. U.S. Pat. No. 10,251,611 describes a freezing of gait cue apparatus which provides a visual cue upon detection of a gait irregularity. US2014/0249600 discloses detection of a patient's gait parameter and providing stimulation by way of electrodes in the brain. Cueing can be applied to different types of gait irregularities, such as hemiplegic gait following a stroke. In these cases, cueing can used to regulate gait variability.

### PROBLEMS WITH THE PRIOR ART

In order to determine the need for cueing or other types of therapy, it is often desirable to determine the severity of FoG, in the form of for example frequency and duration, typically experienced by a patient. However, the prior art does not provide any simple yet reliable method for determining the severity of FoG in a patient.

### SUMMARY

The above described problem is addressed by the claimed FoG analysis device for determining the severity of FoG in a patient. The device preferably comprises a FoG registration interface comprising two thumb-actuated buttons for registering the FoG status of the patient as “FoG” (experiencing FoG) or “no FoG” (not experiencing FoG), where one button indicates “FoG” and the other button indicates “no FoG”. The FoG analysis device is preferably arranged to, while a patient conducts a predefined gait sequence: begin the “FoG” registration by setting the FoG status to “no FoG”; set the FoG status to “FoG” when a user actuates the button indicating “FoG”; set the FoG status to “no FoG” when a user actuates the button indicating “no FoG”; during what remains of the predefined gait sequence, repeat the setting of the FoG status for each change in the patient's FoG status; and based on these registrations of the FoG status, determine the amount of time the FoG status of the patient was registered as FoG as a percentage of the total time for the predefined gait sequence.

The above described problem is further addressed by the claimed method for determining the severity of FoG in a patient. The method preferably comprises:
instructing a patient to conduct a predefined gait sequence; using a FoG analysis device having a FoG registration interface comprising two thumb-actuated buttons for registering the FoG status of the patient as “FoG” (experiencing FoG) or “no FoG” (not experiencing FoG) while the predefined gait sequence is conducted; and based on these registrations of the FoG status, determining the amount of time the FoG status of the patient was registered as “FoG” as a percentage of the total time for the predefined gait sequence. The FoG status is preferably registered by: when a first FoG event occurs, using a predefined “FoG” thumb to actuate the button indicating “FoG”; when the first FoG event stops occurring, using a predefined “no FoG” thumb to actuate the button indicating “no FoG”; and during what remains of the predefined gait sequence, for each change in the patient's FoG status, using the predefined thumbs to register the FoG status as either “FoG” or “no FoG”.

The above described problem is also addressed by the claimed non-transitory computer-readable medium comprising instructions which, when read by a computer, cause the computer to: display a FoG registration interface comprising two thumb-actuated buttons for registering a FoG status of a patient as “FoG” (experiencing FoG) or “no FoG” (not experiencing FoG); allow the FoG status to be registered while a predefined gait sequence is conducted by the patient, by: initially setting the FoG status to “no FoG”; in response to the actuation of the button indicating “FoG”, setting the FoG status to “FoG”; and in response to the actuation of the button indicating “no FoG”, setting the FoG status to “no FoG”; based on these registrations of the FoG status, determine the amount of time the FoG status of the patient was registered as “FoG” as a percentage of the total time for the predefined gait sequence; and output the determined FoG percentage.

This enables a simple yet reliable determination of the severity of FoG episodes in a patient. By the FoG analysis device being designed such that one of the user's thumbs is to activate the “FoG” button and the other is to activate the “no FoG” button, while the device is held in the user's palms, the user is free to carefully observe the patient's gait during the predefined gait sequence and thus observe whether or not a gait abnormality is occurring at any given instant during the predefined gait sequence. Actuation of the buttons can of course instead occur using fingers other than the thumbs.

In embodiments, the buttons are arranged so that a user holding the FoG analysis device in both hands can easily actuate the button indicating “FoG” with a predefined “FoG” thumb, but not with a predefined “no FoG” thumb, and easily actuate the button indicating “no FoG” with the predefined “no FoG” thumb, but not with the predefined “FoG” thumb. This arrangement facilitates for the user to activate the “FoG” and “no FoG” buttons without having to look directly at the buttons, so that the user can focus on carefully observing the patient's gait during the predefined gait sequence and thus observe whether or not a gait abnormality is occurring at any given instant during the predefined gait sequence.

In embodiments, each button is arranged to cover at least the entire portion of the surface area of the FoG registration interface that a typical user is able to actuate with the respective predefined thumb. The spacing between the buttons is preferably also such that the likelihood of the incorrect button being activated is remote.

In embodiments, the FoG registration interface further comprises a START button and a STOP button, and the FoG analysis device calculates the total time for the predefined gait sequence as the time between an actuation of the START button and an actuation of the STOP button.

In embodiments, the determined FoG percentage or FoG duration is output on the FoG registration interface. The determined FoG percentage or FoG duration may alternatively, or additionally, be output in other ways, for example by being transmitted electronically for output by alternative means.

In embodiments, the number of FoG episodes or FoG frequency registered during the predefined gait sequence is output on the FoG registration interface. The determined number of FoG episodes or FoG frequency may alternatively, or additionally, be output in other ways, for example by being transmitted electronically for output by alternative means.

In embodiments, the FoG analysis device is implemented in the form of a smartphone or a tablet computer arranged with FoG registering software, wherein the two buttons of the FoG registration interface are arranged to be within easy reach of the thumbs when a user holds the smartphone or tablet computer in both hands.

The user would typically be a clinician or movement scientist who is interested in evaluating the severity of FoG in a patient.

The determined FoG severity could be used for determining a suitable FoG treatment, for example in the form of cueing methodologies.

The patient could also carry body-worn motion sensors, and the output of these sensors could be correlated with the determined FoG. This could be a way of training a machine learning system to automatically determine FoG.

The device is herein described as a FoG analysis device, but it could easily be adapted by those skilled in the art to analyse other gait abnormalities. The device could thus generally be used to determine the frequency and duration of a different gait artefact during a predefined gait sequence, where the ON button is used for logging those times when this gait artefact is present, and the OFF button is used for logging those times when this gait artefact is absent. The device could, in this manner, be used to determine the frequency and duration of a wide diversity of gait artefacts. These data could be used to support the clinical management of patients around these gait artefacts.

The scope of the invention is defined by the claims, which are incorporated into this section by reference. A more complete understanding of embodiments of the invention will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

FIGS. 1-5 illustrate an example FoG analysis device, in accordance with one or more embodiments described herein.

FIG. 6 schematically illustrates time synchronization between a FoG analysis device and a video recording device, in accordance with one or more embodiments described herein

FIG. 7 schematically illustrates the use of body-worn motion sensors together with a FoG analysis device, in accordance with one or more embodiments described herein.

FIG. 8 schematically illustrates the use of body-worn motion sensors together with a video camera, in accordance with one or more embodiments described herein.

FIG. 9 schematically illustrates an example method for determining the severity of FoG in a patient, in accordance with one or more embodiments described herein.

Embodiments of the present disclosure and their advantages are best understood by referring to the detailed description that follows. It should be appreciated that like reference numerals are used to identify like elements illustrated in one or more of the figures.

### DETAILED DESCRIPTION

In order to determine the severity of FoG in a patient, it is advantageous to use predefined gait sequences. Such predefined gait sequences may be in the form of different types of standardised FoG provocation tests, which have different test conditions. The Ziegler test (described in the article “A new rating instrument to assess festination and freezing gait in Parkinsonian patients”, Mov Disord 25, 1012-1018, by K Ziegler et. al) is one example of a test which is designed to provoke and determine FoG.

A task in the form of a Ziegler FoG score test may in examples comprise various sub-tasks, such Single tasks, Dual Motor tasks, and Dual Motor & Cognitive tasks. Other tests may comprise sub-tasks even more aimed at provoking FoG, such as for example Time Up and Go (TUG) tasks, Doorway tasks and various 360° Turn tasks.

The present disclosure relates generally to methods and devices for determining a severity of FoG in a patient. Embodiments of the disclosed solution are presented in more detail in connection with the figures.

The present disclosure also relates generally to devices and methods for determining a level of other gait abnormalities/gait artefacts associated with neurological and other diseases, for example, time epochs when the patient is walking with and is walking without gait unsteadiness or other specific gait abnormalities/artefacts.

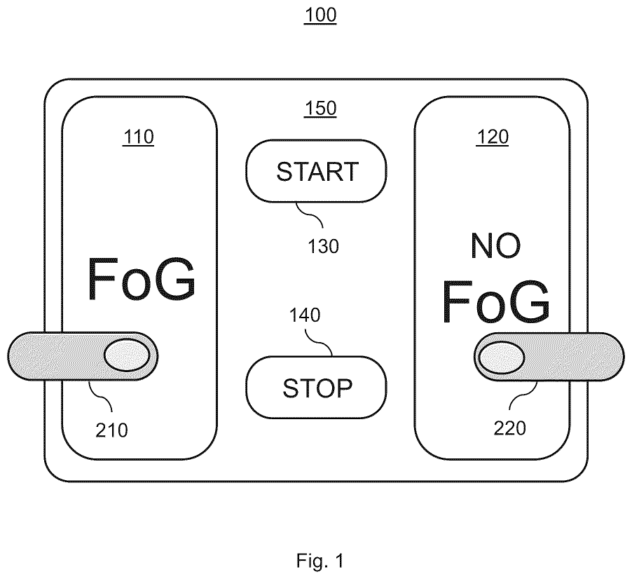

FIGS. 1-5 illustrate an example FoG analysis device 100 for determining a severity of FoG in a patient 250. The illustrated FoG analysis device 100 comprises a FoG registration interface 150 comprising two thumb-actuated buttons 110, 120 for registering the FoG status of the patient 250. One button 110 indicates “FoG” (experiencing FoG) and the other button 120 indicates “no FoG” (not experiencing FoG). The buttons 110, 120 are arranged to be actuated by respective thumbs 210, 220 of a user while a patient 250 conducts a predefined gait sequence. The FoG registration interface 150 may also comprise a START button 130 and a STOP button 140.

The buttons 110, 120 are preferably arranged so that a user holding the FoG analysis device 100 in both hands can easily actuate the button 110 indicating “FoG” with a predefined “FoG” thumb 210, but not with a predefined “no FoG” thumb 220, and easily actuate the button 120 indicating “no FoG” with the predefined “no FoG” thumb 220, but not with the predefined “FoG” thumb 210. The buttons 110, 120 would typically be actuated while the user 200 is carefully observing the patient's gait while they are performing the predefined gait sequences.

A user 200, typically a clinician or movement scientist, may begin the “FoG” registration by entering patient data into the FoG registration interface 150. The patient data comprises an identifier of the patient 250 (such as a name or number), and an indication of the type of test to be performed, for example in the form of a test condition and a task and/or a sub-task. This information may be displayed in the FoG registration interface 150 during the FoG registration.

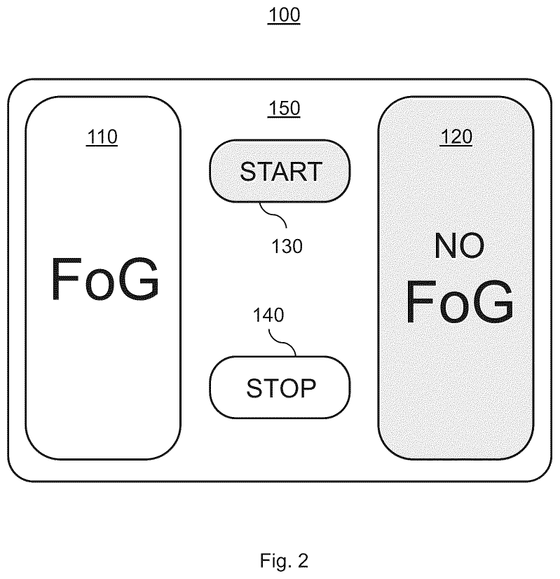

The user 200 then instructs the patient 250 to conduct the predefined gait sequence corresponding to the task or sub-task. The user 200 actuates the START button 130 as the patient 250 begins the predefined gait sequence. At this point, the FoG status is set to “no FoG”, which (as shown in FIG. 2) may be indicated on the FoG registration interface 150.

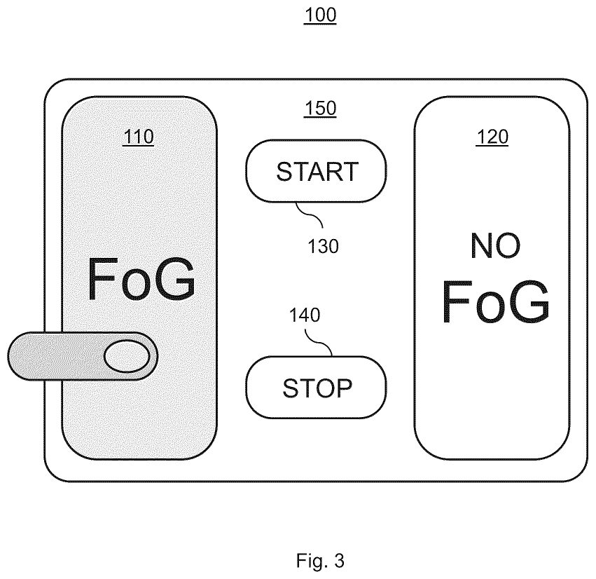

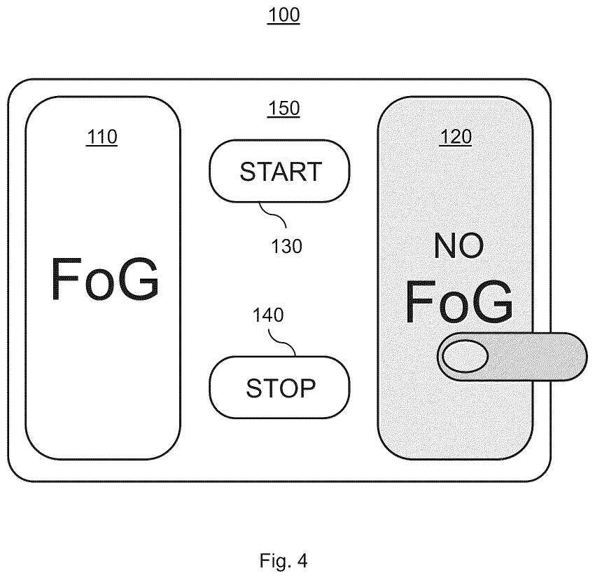

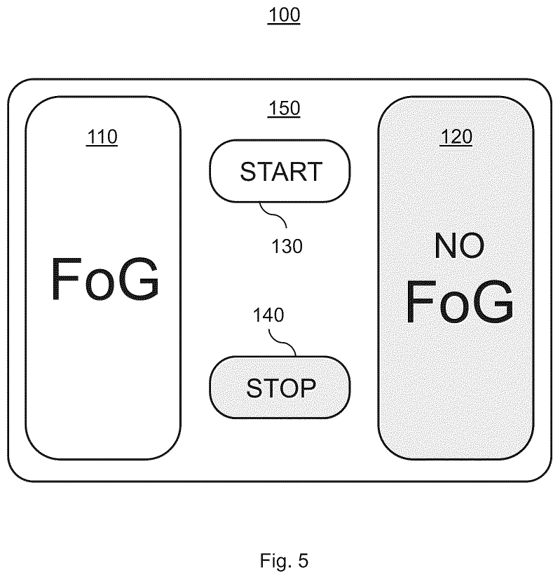

The user 200 continuously monitors the patient 250 while the predefined gait sequence is conducted. When the user 200 observes the onset of a FoG episode, the user 200 actuates the button 110 indicating “FoG” with the predefined “FoG” thumb 210 (in this case the left thumb), which changes the FoG status to “FoG” (as shown in FIG. 3). When the user 200 sees that the FoG episode has ended, the user 200 actuates the button 120 indicating “no FoG” with the predefined “no FoG” thumb 220 (in this case the right thumb), which changes the FoG status to “no FoG” (as shown in FIG. 4). The user 200 in this way keeps actuating the buttons 110, 120 with the thumbs 210, 220 as soon as there is a change in FoG status. When the patient 250 has finished the predefined gait sequence, the user 200 actuates the STOP button 140 (as shown in FIG. 5), and the analysis can begin.

Based on the registrations of the FoG status, the FoG analysis device 100 determines the amount of time the FoG status of the patient 250 was registered as FoG as a percentage of the total time for the predefined gait sequence. This time is typically referred to as the Percentage of Time in FoG (PTF). The total time for the predefined gait sequence may for example be determined as the time between the actuating of the START button 130 and the actuating of the STOP button 140. The percentage in which the FoG status of the patient 250 was registered as FoG, or the FoG duration, may then be output, for example on the FoG registration interface 150. The determined FoG percentage or FoG duration may alternatively, or additionally, be output in other ways, for example by being transmitted electronically for output by alternative means.

The FoG analysis device 100 may also determine the number of FoG episodes or the FoG frequency registered during the predefined gait sequence, and output also this, for example on the FoG registration interface 150. The determined number of FoG episodes or FoG frequency may alternatively, or additionally, be output in other ways, for example by being transmitted electronically for output by alternative means.

Since the user 200 needs to monitor the patient 250 during the whole predefined gait sequence, the buttons 110, 120 are preferably very large and spaced sufficiently far apart so that the user 200 can easily actuate the correct button 110, 120 even when only having peripheral vision of the FoG registration interface 150. Each button 110, 120 is preferably arranged to cover at least the entire portion of the surface area of the FoG registration interface 150 that a typical user 200 is able to actuate with the respective predefined thumb 210, 220, so that the user 200 does not have to watch the FoG registration interface 150 in order to actuate the correct button 110, 120. The spacing between the buttons 110, 120 is preferably also such that the likelihood of the incorrect button 110, 120 being activated is remote. A user 200, such as a clinician or movement scientist, who registers FoG status using such a FoG registration interface 150 will be able to reliably determine the severity of FoG in a patient 250.

The determination of FoG percentage or FoG duration, or number of FoG episodes or FoG frequency, may be done for a number of tests in combination. If the patient 250 is instructed to perform a number of different tasks or sub-tasks, the combined FoG percentage or FoG duration, or number of FoG episodes or FoG frequency, for all of the tasks or sub-tasks may be determined.

The buttons 110, 120 may be colour-coded when activated, so that it is easy for the user 200 to see the current FoG status. The “FoG” button 110 could for example be red when active (and e.g. white when inactive), and the “no FoG” button 120 green when active (and e.g. white when inactive).

If the user 200 actuates a button 110, 120 that does not change the FoG status, this will preferably be ignored by the system. In this way, the user 200 does not have to keep track of the current FoG status, the user 200 can simply actuate the “FoG” button 110 when observing FoG, and actuate the “no FoG” button 120 when not observing FoG.

The current FoG status may be indicated on the FoG registration interface 150 by the active button 110, 120 being “turned on”, for example by the colour being changed. In this case, actuating the “FoG” button 110 preferably automatically “switches off” the “no FoG” button 120, and actuating the “no FoG” button 120 preferably automatically “switches off” the “FoG” button 110.

The FoG analysis device 100 may be implemented in the form of a smartphone or a tablet computer arranged with FoG registering software. The FoG registration interface 150 is in this case preferably configured so that the two buttons 110, 120 of the FoG registration interface 150 are arranged to be within easy reach of the thumbs 210, 220 when a user 200 holds the smartphone or tablet computer in both hands.

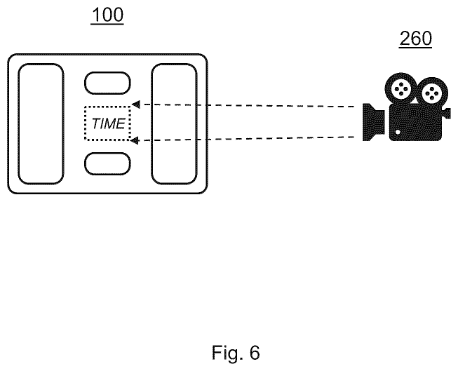

The FoG analysis device 100 can support the capability for a video recording device 260 to capture a time stamp from the FoG analysis device 100, so that time synchronization is enabled between the FoG analysis device 100 and the video recording device 260. One method of achieving this by the video recording device 260 capturing a time stamp from the FoG analysis device 100 at the beginning of the recording session is schematically illustrated in FIG. 6. Time synchronization between the FoG analysis device 100 and the video recording device 260 may be used to validate the FoG analysis device 100 by having a clinician or movement scientist analyse the video of the predefined gait sequence via frame-by-frame post-processing. This gold-standard method may be used to determine the frequency and duration of freezing episodes (or other gait abnormalities) occurring during the predefined gait sequence, as a method of validating the output from the FoG analysis device 100.

The FoG analysis device 100 may support the capability to time-synchronise with the capture of patient body-worn motion sensor data simultaneously collected from the patient 250 as they are completing the predefined gait sequence. This could be a way of training a machine learning system to accurately determine FoG based on motion sensor data, and validate AI-based gait abnormality detection algorithms.

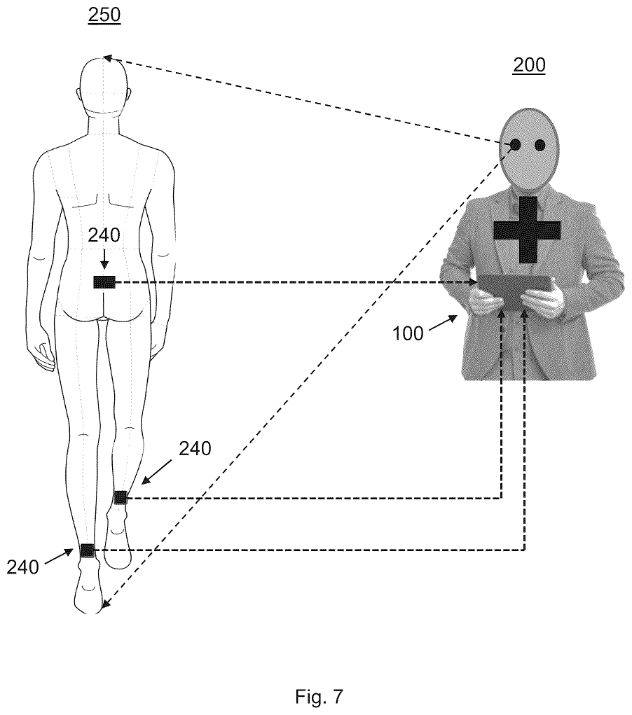

FIG. 7 schematically illustrates the use of body-worn motion sensors 240 together with a FoG analysis device 100. While simultaneously operating the FoG analysis device 100, the user 200 carefully observes all aspects of the patient's locomotion while completing a predefined gait sequence and at each instant determines whether a gait abnormality is occurring. Body-worn motion sensors 240 may be located at multiple body locations including, but not limited to, shins, feet, shanks, thighs, and/or the lower back. The body-worn motion sensors 240 may be wirelessly connected to the FoG analysis device 100, for example via Bluetooth. This may enable the time-synchronisation.

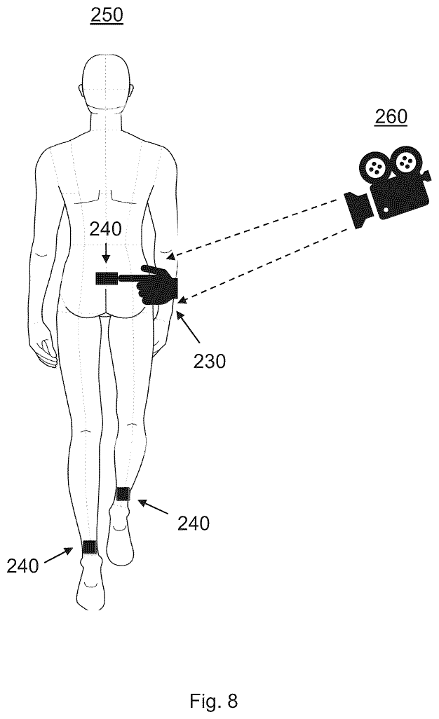

Motion sensor data could also be time-synchronised with video recording by a video recording device 260. One method of achieving this by the video recording device 260 capturing the hand 230 of the user 200 physically tapping (either directly or via a tapping device, such as a pen) a body-worn motion sensor 240 (to generate a motion response on the sensor) to provide time synchronization between the video recording device 260 and the body-worn motion sensor signals is schematically illustrated in FIG. 8. The video recording device 260 may capture multiple taps on each body-worn motion sensor 240, so that there is time synchronization between the video recording device 260 and each body-worn motion sensor 240.

The body-worn motion sensors 240 may thus have two training sources for the development of AI-based gait abnormality detection algorithms: recordings by one or more video recording devices 260, and the output from the FoG analysis device 100. These training sources may each be used alone, or in combination.

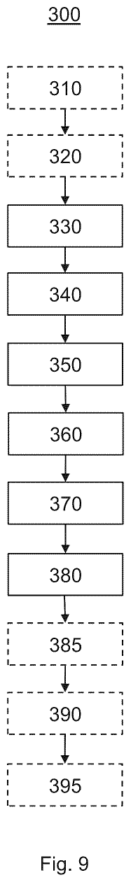

FIG. 9 schematically illustrates a method 300 for determining a severity of FoG in a patient 250. The method 300 may include:
Step 330: instructing a patient 250 to conduct a predefined gait sequence.
Step 340: using a FoG analysis device 100 having a FoG registration interface 150 comprising two thumb-actuated buttons 110, 120 for registering the FoG status of the patient 250 as “FoG” (experiencing FoG) or “no FoG” (not experiencing FoG) while the predefined gait sequence is conducted.
Step 350: when a first FoG event occurs, using a predefined “FoG” thumb 210 to actuate the button 110 indicating “FoG”.
Step 360: when the first FoG event stops occurring, using the predefined “no FoG” thumb 220 to actuate the button 120 indicating “no FoG”.
Step 370: during what remains of the predefined gait sequence, for each change in the patient's FoG status, using the predefined thumbs 210, 220 to register the FoG status as either “FoG” or “no FoG”.
Step 380: based on these registrations of the FoG status, determining the amount of time the FoG status of the patient 250 was registered as “FoG” as a percentage of the total time for the predefined gait sequence.

This enables a simple yet reliable determination of the severity of FoG episodes in a patient 250. By the FoG analysis device 100 being designed such that one of the user's thumbs 210 is to activate the “FoG” button 110 and one 220 is to activate the “no FoG” button 120, while the FoG analysis device 100 is held in the user's palms, the user 200 is free to carefully observe the patient's gait during the predefined gait sequence and thus observe whether or not a gait abnormality is occurring at any given instant during the predefined gait sequence.

In embodiments, the buttons 110, 120 are arranged so that a user 200 holding the FoG analysis device 100 in both hands can easily actuate the button 110 indicating “FoG” with a predefined “FoG” thumb 210, but not with a predefined “no FoG” thumb 220, and easily actuate the button 120 indicating “no FoG” with the predefined “no FoG” thumb 220, but not with the predefined “FoG” thumb 210.

In embodiments, each button 110, 120 is arranged to cover at least the entire portion of the surface area of the FoG registration interface 150 that a typical user 200 is able to actuate with the respective predefined thumb 210, 220. The spacing between the buttons 110, 120 is preferably also such that the likelihood of the incorrect button 110, 120 being activated is remote.

The method 300 may further include one or more of:
Step 310: arranging the FoG analysis device 100 to be a smartphone or a tablet computer arranged with FoG registering software.
Step 320: arranging the two buttons 110, 120 of the FoG registration interface 150 to be within easy reach of the thumbs 210, 220 when a user 200 holds the smartphone or tablet computer in both hands.
Step 385: calculating the total time for the predefined gait sequence as the time between an actuation of the START button 130 and an actuation of the STOP button 140 (in embodiments where the FoG registration interface 150 comprises a START button 130 and a STOP button 140).
Step 390: outputting the determined FoG percentage or FoG duration on the FoG registration interface 150. The determined FoG percentage or FoG duration may alternatively, or additionally, be output in other ways, for example by being transmitted electronically for output by alternative means.
Step 395: outputting the number of FoG episodes or FoG frequency registered during the predefined gait sequence on the FoG registration interface 150. The determined number of FoG episodes or FoG frequency may alternatively, or additionally, be output in other ways, for example by being transmitted electronically for output by alternative means.

The above steps may be effected in any order that makes technical sense. Some of the steps may also be effected simultaneously with each other.

A non-transitory computer-readable medium may comprise instructions which, when read by a computer, cause the computer to: display a FoG registration interface 150 comprising two thumb-actuated buttons for registering a FoG status of a patient 250 as “FoG” (experiencing FoG) or “no FoG” (not experiencing FoG); allow the FoG status to be registered while a predefined gait sequence is conducted by the patient 250, by:
initially setting the FoG status to “no FoG”; in response to the actuation of the button indicating “FoG”, setting the FoG status to “FoG”; and in response to the actuation of the button indicating “no FoG”, setting the FoG status to “no FoG”; based on these registrations of the FoG status, determine the amount of time the FoG status of the patient was registered as “FoG” as a percentage of the total time for the predefined gait sequence; and output the determined FoG percentage.

This enables a simple yet reliable determination of the severity of FoG episodes in a patient 250. By the FoG analysis device 100 being designed such that one of the user's thumbs 210 is to activate the “FoG” button 110 and one 220 is to activate the “no FoG” button 120, while the FoG analysis device 100 is held in the user's palms, the user 200 is free to carefully observe the patient's gait during the predefined gait sequence and thus observe whether or not a gait abnormality is occurring at any given instant during the predefined gait sequence.

The instructions may further cause the FoG registration interface to have the buttons arranged so that a user holding the FoG analysis device in both hands can easily actuate the button indicating “FoG” with a predefined “FoG” thumb, but not with a predefined “no FoG” thumb, and easily actuate the button indicating “no FoG” with the predefined “no FoG” thumb, but not with the predefined “FoG” thumb.

The instructions may further cause the FoG registration interface to have each button cover at least the entire portion of the surface area of the FoG registration interface that a typical user is able to actuate with the respective predefined thumb. The spacing between the buttons 110, 120 is preferably also such that the likelihood of the incorrect button 110, 120 being activated is remote.

The instructions may cause the FoG registration interface to comprise a START button 130 and a STOP button 140, and the instructions may further cause the computer to calculate the total time for the predefined gait sequence as the time between an actuation of a START button 130 and an actuation of a STOP button 140.

The instructions may further cause the computer to output the determined FoG percentage or FoG duration on the FoG registration interface 150. The determined FoG percentage or FoG duration may alternatively, or additionally, be output in other ways, for example by being transmitted electronically for output by alternative means.

The instructions may further cause the computer to output the number of FoG episodes or FoG frequency registered during the predefined gait sequence on the FoG registration interface 150. The determined number of FoG episodes or FoG frequency may alternatively, or additionally, be output in other ways, for example by being transmitted electronically for output by alternative means.

The instructions may further cause a smartphone or tablet computer to arrange the two buttons of the FoG registration interface 150 to be within easy reach of the thumbs when a user holds the smartphone or tablet computer in both hand (in embodiments where the computer is a smartphone or a tablet computer).

The foregoing disclosure is not intended to limit the present invention to the precise forms or particular fields of use disclosed. It is contemplated that various alternate embodiments and/or modifications to the present invention, whether explicitly described or implied herein, are possible in light of the disclosure. Accordingly, the scope of the invention is defined only by the claims.

1. A FoG analysis device for determining a severity of Freezing Of Gait (FoG) in a patient, the device comprising a FoG registration interface comprising two thumb-actuated buttons for registering the FoG status of the patient as “FoG” (experiencing FoG) or “no FoG” (not experiencing FoG), where one button indicates “FoG” and the other button indicates “no FoG”, the FoG analysis device being arranged to, while a patient conducts a predefined gait sequence:
begin the “FoG” registration by setting the FoG status to “no FoG”;
set the FoG status to “FoG” when a user actuates the button indicating “FoG”;
set the FoG status to “no FoG” when a user actuates the button indicating “no FoG”;
during what remains of the predefined gait sequence, repeat the setting of the FoG status for each change in the patient's FoG status; and
based on these registrations of the FoG status, determine the amount of time the FoG status of the patient was registered as FoG as a percentage of the total time for the predefined gait sequence.

2. A FoG analysis device according to claim 1, wherein the buttons are arranged so that a user holding the FoG analysis device in both hands can easily actuate the button indicating “FoG” with a predefined “FoG” thumb, but not with a predefined “no FoG” thumb, and easily actuate the button indicating “no FoG” with the predefined “no FoG” thumb, but not with the predefined “FoG” thumb.

3. A FoG analysis device according to claim 1, wherein each button is arranged to cover at least the entire portion of the surface area of the FoG registration interface that a typical user is able to actuate with the respective predefined thumb.

4. A FoG analysis device according to claim 1, wherein the FoG registration interface further comprises a START button and a STOP button, and the FoG analysis device is arranged to calculate the total time for the predefined gait sequence as the time between an actuation of the START button and an actuation of the STOP button.

5. A FoG analysis device according to claim 1, further arranged to output the determined FoG percentage on the FoG registration interface.

6. A FoG analysis device according to claim 1, further arranged to output the number of FoG episodes registered during the predefined gait sequence on the FoG registration interface.

7. A FoG analysis device according to claim 1, implemented in the form of a smartphone or a tablet computer arranged with FoG registering software, wherein the two buttons of the FoG registration interface are arranged to be within easy reach of the thumbs when a user holds the smartphone or tablet computer in both hands.

8. A method for determining a severity of Freezing Of Gait (FoG) in a patient, the method comprising:
instructing a patient to conduct a predefined gait sequence;
using a FoG analysis device having a FoG registration interface comprising two thumb-actuated buttons for registering the FoG status of the patient as “FoG” (experiencing FoG) or “no FoG” (not experiencing FoG) while the predefined gait sequence is conducted, wherein the FoG status is registered by:
when a first FoG event occurs, using a predefined “FoG” thumb to actuate the button indicating “FoG”;
when the first FoG event stops occurring, using the predefined “no FoG” thumb to actuate the button indicating “no FoG”; and
during what remains of the predefined gait sequence, for each change in the patient's FoG status, using the predefined thumbs to register the FoG status as either “FoG” or “no FoG”; and
based on these registrations of the FoG status, determining the amount of time the FoG status of the patient was registered as “FoG” as a percentage of the total time for the predefined gait sequence.

9. A method according to claim 8, wherein the buttons are arranged so that a user holding the FoG analysis device in both hands can easily actuate the button indicating “FoG” with a predefined “FoG” thumb, but not with a predefined “no FoG” thumb, and easily actuate the button indicating “no FoG” with the predefined “no FoG” thumb, but not with the predefined “FoG” thumb.

10. A method according to claim 8, wherein each button is arranged to cover at least the entire portion of the surface area of the FoG registration interface that a typical user is able to actuate with the respective predefined thumb.

11. A method according to claim 8, wherein the FoG registration interface further comprises a START button and a STOP button, the method further comprising calculating the total time for the predefined gait sequence as the time between an actuation of the START button and an actuation of the STOP button.

12. A method according to claim 8, further comprising outputting the determined FoG percentage on the FoG registration interface.

13. A method according to claim 8, further comprising outputting the number of FoG episodes registered during the predefined gait sequence on the FoG registration interface.

14. A method according to claim 8, further comprising arranging the FoG analysis device to be a smartphone or a tablet computer arranged with FoG registering software, and arranging the two buttons of the FoG registration interface to be within easy reach of the thumbs when a user holds the smartphone or tablet computer in both hands.

15. A non-transitory computer-readable medium comprising instructions which, when read by a computer, cause the computer to:
display a FoG registration interface comprising two thumb-actuated buttons for registering a FoG status of a patient as “FoG” (experiencing FoG) or “no FoG” (not experiencing FoG);
allow the FoG status to be registered while a predefined gait sequence is conducted by the patient, by:
initially setting the FoG status to “no FoG”;
in response to the actuation of the button indicating “FoG”, setting the FoG status to “FoG”; and
in response to the actuation of the button indicating “no FoG”, setting the FoG status to “no FoG”;
based on these registrations of the FoG status, determine the amount of time the FoG status of the patient was registered as “FoG” as a percentage of the total time for the predefined gait sequence; and
output the determined FoG percentage.

16. A non-transitory computer-readable medium according to claim 15, wherein the instructions cause the FoG registration interface to have the buttons arranged so that a user holding the FoG analysis device in both hands can easily actuate the button indicating “FoG” with a predefined “FoG” thumb, but not with a predefined “no FoG” thumb, and easily actuate the button indicating “no FoG” with the predefined “no FoG” thumb, but not with the predefined “FoG” thumb.

17. A non-transitory computer-readable medium according to claim 15, wherein the instructions cause the FoG registration interface to have each button cover at least the entire portion of the surface area of the FoG registration interface that a typical user is able to actuate with the respective predefined thumb.

18. A non-transitory computer-readable medium according to claim 15, wherein the instructions cause the FoG registration interface to comprise a START button and a STOP button, and the instructions further cause the computer to calculate the total time for the predefined gait sequence as the time between an actuation of the START button and an actuation of the STOP button.

19. A non-transitory computer-readable medium according to claim 15, wherein the instructions further cause the computer to output the determined FoG percentage on the FoG registration interface.

20. A non-transitory computer-readable medium according to claim 15, wherein the instructions further cause the computer to output the number of FoG episodes registered during the predefined gait sequence on the FoG registration interface.

21. A non-transitory computer-readable medium according to claim 15, wherein the computer is a smartphone or a tablet computer, and the instructions further cause the smartphone or tablet computer to arrange the two buttons of the FoG registration interface to be within easy reach of the thumbs when a user holds the smartphone or tablet computer in both hands.
